(19) United States
(12) Patent Application Publication
(10) Pub. No.: US 2019/0021578 A1
(43) Pub. Date: Jan. 24, 2019

(54) ENDOSCOPE, OPTICAL MACHINE CONNECTING DEVICE AND METHOD FOR MODIFYING TWO-DIMENSIONAL ENDOSCOPE SYSTEM

(71) Applicant: KANGHONG (ZHUHAI) DEVELOPMENT CO., LTD., Tangjiawan Town Zhuhai (CN)

(72) Inventors: Shanyun HU, Tangjiawan Town Zhuhai (CN); Peng LIU, Tangjiawan Town Zhuhai (CN)

(21) Appl. No.: 16/070,983

(22) PCT Filed: Apr. 11, 2016
(86) PCT No.: PCT/CN2016/078949

§ 371 Date: Jul. 18, 2018

(30) Foreign Application Priority Data
Jan. 19, 2016 (CN) 201610033568.2

(51) Int. Cl. A61B 1/00; A61B 1/04; G02B 23/24

(52) U.S. Cl. CPC A61B 1/00009; A61B 1/04; G02B 23/2407; A61B 1/07; A61B 1/00126; A61B 1/00163; A61B 1/00096; A61B 1/00013

(57) ABSTRACT

An endoscope, an optical machine connecting device and a method for modifying a two-dimensional endoscope system are provided. The endoscope comprises an optical machine connecting unit, an insert tube (1), and a first lens (3) and a second lens (4) that are integrated into the insert tube (1). The optical machine connecting unit comprises an optical deflection assembly (5) and a connecting assembly (7). The optical deflection assembly (5) is used for changing a spacing between optical axes of a first light beam (021) and a second light beam (031) received and projected by the first lens (3) and the second lens (4), so as to match an image sensor (012) in a camera connector (01) of the two-dimensional endoscope system. The connecting assembly (7) is used for forming a butt joint between the endoscope and the camera connector (01), and limiting a spacing between the optical deflection assembly (5) and the image sensor (012). The optical machine connecting device corresponds to the optical machine connecting unit in the endoscope. In the modifying method, an existing two-dimensional endoscope system is upgraded to a three-dimensional endoscope system by using the endoscope by a selection step (S1), a butt-jointing and limiting step (S2) and a step (S3-S4) of upgrading and replacing a rear-end processing device.

### FIELD OF THE INVENTION

The present invention relates to an endoscope, an optical machine connecting device and a method for modifying an existing two-dimensional endoscope system by using the endoscope. The present invention is based on Chinese Invention Patent Application No. 201610033568.2 filed on Jan. 19, 2016, the disclosure of which is incorporated herein as a reference that is closely related to the present invention.

### BACKGROUND OF THE INVENTION

Recently, with the development and popularization of minimally invasive surgery, medical endoscope systems are extensively applied in many fields such as orthopedics.

Chinese Patent Publication No. CN104935915A disclosed a three-dimensional endoscope, including an insert tube and a three-dimensional imaging unit, wherein the three-dimensional imaging unit includes an image sensor and a first lens and a second lens that are integrated into the insert tube; the first lens and the second lens are used for capturing images of the same scene at the same moment to acquire a first image and a second image having a parallax therebetween, and projecting the first image and the second image onto a target surface of the same image sensor for imaging.

Compared with the conventional two-dimensional endoscope system, a three-dimensional endoscope system containing the three-dimensional endoscope can truly restore the perspective field of view of the surgery and have an amplification function, which facilitates the discovery of lesion sites among various internal organs so as to accurately cut off and reconstruct the lesion sites, especially in a surgery with multiple anatomical levels, complicated blood vessels and large operation difficulty. The three-dimensional endoscope system assists doctors to better perform an operation by presenting the feeling of depth in the real surgery field.

### SUMMARY OF THE INVENTION

### Technical problems

At present, quite a few hospitals still use two-dimensional endoscope systems. If these two-dimensional endoscope systems are replaced with three-dimensional endoscope systems, it will not only cost a lot of money, but also cause a large number of idle facilities. In addition to the two-dimensional endoscope systems used for human medical treatment, two-dimensional endoscope systems used for animal medical and industrial applications also have the above upgrading problem.

### Technical solutions

A main objective of the present invention is to provide an endoscope available for modifying an existing two-dimensional endoscope system into a three-dimensional endoscope system.

Another objective of the present invention is to provide an optical machine connecting device for the endoscope.

Yet another objective of the present invention is to provide a method for upgrading an existing two-dimensional endoscope system to a three-dimensional endoscope system by using the above endoscope.

To achieve the above main objectives, the endoscope provided by the present invention includes an optical machine connecting unit, an insert tube, and a first lens and a second lens that are integrated into the insert tube. The optical machine connecting unit includes an optical deflection assembly and a connecting assembly. The optical deflection assembly includes a first prism and second prism. In a travelling direction of light beams in the endoscope, the first prism is located downstream of the first lens, and the second prism is located downstream of the second lens. The first prism is used for reflecting a first light beam received and projected by the first lens, and the second prism is used for reflecting a second light beam received and projected by the second lens, with a spacing between optical axes of the first light beam and the second light beam being changed, so as to match an image sensor in a camera connector of a two-dimensional endoscope system. The connecting assembly is used for forming a butt joint between the endoscope and the camera connector and limiting a spacing between the optical deflection assembly and the image sensor.

As a specific embodiment, both the first prism and the second prism are parallelogram prisms. The optical deflection assembly is simple in structure and low in cost.

As another specific embodiment, the first prism includes a first reflection plane, a second reflection plane and a third reflection plane, the second reflecting plane is parallel to the optical axis of the first light beam, and the first reflection plane and the third reflection plane are located on the same side of the second reflection plane. In the travelling direction of light beams in the endoscope, the third reflection plane is located downstream of the first reflection plane, the spacing between the first reflection plane and the second reflection plane decreases gradually, the spacing between the third reflection plane and the second reflection plane increases gradually, and an included angle between the first reflection plane and the second reflection plane is equal to that between the third reflection plane and the second reflection plane. The second prism includes a fourth reflection plane, a fifth reflection plane and a sixth reflection plane, the fifth reflection plane is parallel to the optical axis of the second light beam, and the fourth reflection plane and the sixth reflection plane are located on the same side of the fifth reflection plane. In the travelling direction of light beams in the endoscope, the sixth reflection plane is located downstream of the fourth reflection plane, the spacing between the fourth reflection plane and the fifth reflection plane decreases gradually, the spacing between the sixth reflection plane and the fifth reflection plane increases gradually, and an included angle between the fourth reflection plane and the fifth reflection plane is equal to that between the sixth reflection plane and the fifth reflection plane. Accordingly, it is convenient for controlling the transverse dimension of the optical deflection assembly.

As a more specific embodiment, the first prism comprises a first front prism and a first rear prism; the first reflection plane is located on the first front prism, and the second reflection plane and the third reflection plane are located on the first rear prism; butt joint surfaces of the first front prism and the first rear prism are fixedly connected by glue; and anti-reflection films are coated on the butt joint surfaces of the first front prism and the first rear prism. The second prism comprises a second front prism and a second rear prism; the fourth reflection plane is located on the second front prism, and the fifth reflection plane and the sixth reflection plane are located on the second rear prism; butt joint surfaces of the second front prism and the second rear prism are fixedly connected by glue; and, anti-reflection films are coated on the butt joint surfaces of the second front prism and the second rear prism.

As a preferred embodiment, the connecting assembly includes a connecting collar and a positioning screw. The connecting collar is formed with an inner shoulder on an inner side of one end of the connecting collar, while the other end thereof is detachably and fixedly connected to the camera connector. The connecting collar is provided with a screw hole matched with the positioning screw on a side wall of the one end thereof. The one end of the connecting collar is sheathed on one end of the insert tube, and the insert tube can rotate about an axis of the connecting collar relative to the connecting collar. When the endoscope is butt-jointed with the camera connector, an end face of the one end of the insert tube is rested against the inner shoulder. The assembly process is simple.

As another preferred embodiment, the connecting assembly includes a fixed connecting ring and a rotary connecting ring. One end of the rotary connecting ring is buckled on one end of the fixed connecting ring and can rotate about its own axis relative to the fixed connecting ring. The inner diameter of the fixed connecting ring is less than that of the rotary connecting ring. The other end of the fixed connecting ring is fixedly connected to the camera connector by threads. The insert tube is formed with external threads on one end of the insert tube, and the rotary connecting ring is formed with internal threads matched with the external threads on an inside wall of the other end of the rotary connecting ring. The direction of turning of the internal threads is opposite to that of the threads formed on the other end of the fixed connecting ring. When the endoscope is butt-jointed with the camera connector, an end face of the one end of the insert tube is rested against an end face of the one end of the fixed connecting ring.

To achieve the another objective mentioned above, the present invention provides an optical machine connecting device for an endoscope, which is used for connecting the endoscope with a camera connector of a two-dimensional endoscope system. The endoscope includes an insert tube, and a first lens and a second lens that are integrated into the insert tube. The optical machine connecting device includes an optical deflection assembly and a connecting assembly. The optical deflection assembly includes a first prism and second prism. In a travelling direction of light beams in the endoscope, the first prism is located downstream of the first lens, and the second prism is located downstream of the second lens. The first prism is used for reflecting a first light beam received and projected by the first lens, and the second prism is used for reflecting a second light beam received and projected by the second lens, with a spacing between optical axes of the first light beam and the second light beam being changed, so as to match an image sensor in a camera connector of a two-dimensional endoscope system. The connecting assembly is used for forming a butt joint between the endoscope and the camera connector and limiting a spacing between the optical deflection assembly and the image sensor.

As a specific embodiment, the connecting assembly includes a connecting collar; one end of the connecting collar is fixedly connected to one end of the insert tube by threads, while the other end thereof is fixedly connected to the camera connector by threads; and both the first prism and the second prism are mounted within the connecting collar.

To achieve the yet another objective, the present invention provides a method for modifying a two-dimensional endoscope system having a two-dimensional endoscope, a camera connector and a rear-end processing device, including a selection step, a butt-jointing and limiting step and a step of upgrading and replacing the rear-end processing device. The selection step is as follows: selecting a three-dimensional endoscope matched with the camera connector to replace the existing two-dimensional endoscope, the three-dimensional endoscope being the endoscope described in any one of the above technical solutions. The butt-jointing and limiting step is as follows: butt-jointing the three-dimensional endoscope with the camera connector by the connecting assembly, and limiting a spacing between the optical deflection assembly and an image sensor in the camera connector.

### Beneficial effects

In the endoscope provided by the present invention, with the optical machine connecting unit, the spacing between optical axes of light beams in the first image and the second image having a parallax therebetween acquired by the first lens and the second lens at the same moment is changed, and the first image and the second image are projected onto mutually separated first photosensitive region and second photosensitive region on the target surface of the image sensor in the camera connector for the two-dimensional endoscope system. Accordingly, by the modifying method provided by the present invention, the existing two-dimensional endoscope system can be modified into the three-dimensional endoscope system, thereby reducing the upgrade cost and decreasing the number of idle facilities in the existing two-dimensional endoscope system.

In addition, the optical machine connecting device provided by the present invention can connect the insert tube, into which the first lens and the second lens are integrated, to the camera connector. Accordingly, it is only necessary to purchase a small number of insert tubes into which the first lens and the second lens are integrated and a plurality of optical machine connecting devices to upgrade the two-dimensional endoscope systems having images sensors of various different dimensions, so that the upgrade cost is reduced.

### BRIEF DESCRIPTION OF THE DRAWINGS

FIG. 1 is a schematic perspective view of a camera connector of an existing two-dimensional endoscope system;

FIG. 2 is a schematic structure view of the camera connector shown in FIG. 1;

FIG. 3 is a schematic perspective view of a first embodiment of an endoscope according to the present invention with a connecting assembly omitted, viewed from a first perspective;

FIG. 4 is a schematic perspective view of the first embodiment of the endoscope according to the present invention with a connecting assembly omitted, viewed from a second perspective;

FIG. 5 is an axially sectional view of the first embodiment of the endoscope according to the present invention with a connecting assembly omitted;

FIG. 6 is a schematic perspective view of the connecting assembly in the first embodiment of the endoscope according to the present invention;

FIG. 7 is an axially sectional view of the connecting assembly in the first embodiment of the endoscope according to the present invention;

FIG. 8 is a schematic view of light paths in an optical deflection assembly in the first embodiment of the endoscope according to the present invention;

FIG. 9 is a flowchart of a method for modifying an existing two-dimensional endoscope system by using the first embodiment of the endoscope according to the present invention;

FIG. 10 is a schematic structure view of an image sensor in the camera connector shown in FIG. 1;

FIG. 11 is a first state view of the selection of a prism structure in a selection step in the method for modifying an existing two-dimensional endoscope system by using the first embodiment of the endoscope according to the present invention;

FIG. 12 is a second state view of the selection of a prism structure in the selection step in the method for modifying an existing two-dimensional endoscope system by using the first embodiment of the endoscope according to the present invention;

FIG. 13 is a third state view of the selection of a prism structure in the selection step in the method for modifying an existing two-dimensional endoscope system by using the first embodiment of the endoscope according to the present invention;

FIG. 14 is a schematic view of a process of assembling the first embodiment of the endoscope according to the present invention and a camera connector;

FIG. 15 is a structural block view of a three-dimensional endoscope system after the two-dimensional endoscope system is upgraded to the three-dimensional endoscope system by using the first embodiment of the endoscope according to the present invention;

FIG. 16 is a schematic perspective view of a connecting assembly in a second embodiment of the endoscope according to the present invention;

FIG. 17 is an axially sectional view of the connecting assembly in the second embodiment of the endoscope according to the present invention;

FIG. 18 is a schematic view of a process of assembling the second embodiment of the endoscope according to the present invention and a camera connector;

FIG. 19 is a first schematic view of light paths in a first prism in a fourth embodiment of the endoscope according to the present invention;

FIG. 20 is a second schematic view of light paths in the first prism in the fourth embodiment of the endoscope according to the present invention;

FIG. 21 is a schematic perspective view of a fifth embodiment of the endoscope according to the present invention with a connecting assembly omitted; and

FIG. 22 is a schematic structure view of a sixth embodiment of the endoscope according to the present invention.

The present invention will be further described below by embodiments with reference to the accompanying drawings.

### DETAILED DESCRIPTION OF THE EMBODIMENTS

The main concept of the present invention is to provide a three-dimensional endoscope which can be matched with a camera connector of an existing two-dimensional endoscope system to upgrade the existing two-dimensional endoscope system to a three-dimensional endoscope system, so that the cost for upgrading and replacement is reduced and the number of idle facilities in the existing two-dimensional endoscope system is decreased. It mainly relates to the structure of an optical machine connecting portion for connecting the endoscope to the camera connector in the existing two-dimensional endoscope system, and the structure of other portions is designed according to the existing products.

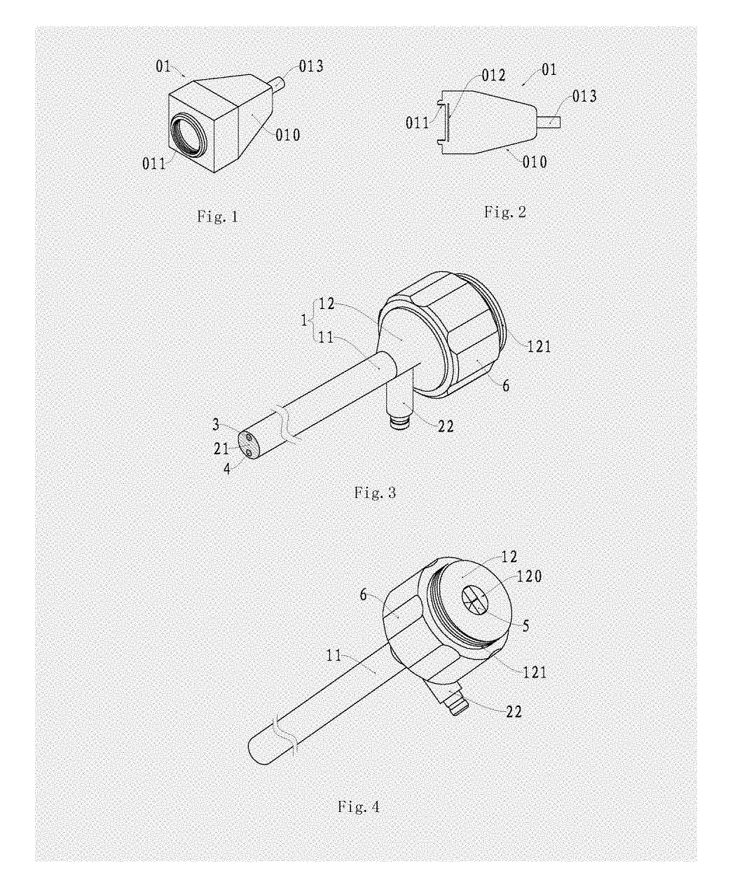

As shown in FIGS. 1 and 2, the camera connector 01 is a camera connector commonly used in an existing two-dimensional medical endoscope system. A base body 010 of the camera connector 01 is provided with, an internally threaded interface 011 at a front end of the base body 010, an image sensor 012 within an inner cavity of the base body 010, and a signal output terminal 013 at a rear end of the base body 010. The image sensor 012 is a CCD sensor. An endoscope matched with the image sensor projects the received light beam onto a target surface of the image sensor 012, and then outputs electrical signals through the signal output terminal 013.

In the following embodiments, the present invention will be described taking a medical endoscope matched with the camera connector 01 as example; however, the connection interface of the camera connector, the interface for connecting the endoscope to the camera connector and the structure of the endoscope are not limited to the following embodiments.

### First Embodiment of the Endoscope

Referring to FIGS. 3-7, the endoscope comprises an insert tube 1; a first lens 3, a second lens 4 and an illumination optical fiber 21 that are all integrated into a front tube 11 of the insert tube 1; an optical deflection assembly 5 arranged within an inner cavity 120 of a rear tube 12 of the insert tube 1; a focusing ring 6 arranged on a periphery of the rear tube 12 of the insert tube 1; an optical fiber interface 22 provided at a junction of the front tube 11 with the rear tube 12; and a connecting assembly 7. The optical deflection assembly 5 and the connecting assembly 7 form an optical machine connecting unit in this embodiment.

Referring to FIGS. 3-5, the first lens 3 comprises a protective casing, a first imaging lens 31, a first set of focusing lens 33 and an image transmitter arranged between the first imaging lens 31 and the first set of focusing lens 33. The first imaging lens 31, the first set of focusing lens 33 and the image transmitter are all disposed within the protective casing. The image transmitter comprises a plurality of image transmitting columns 32. The second lens 4 comprises a protective casing, a second imaging lens 41, a second set of focusing lens 43 and an image transmitter arranged between the second imaging lens 41 and the second set of focusing lens 43. The second imaging lens 41, the second set of focusing lens 43 and the image transmitter are all disposed within the protective casing. The image transmitter comprises a plurality of image transmitting columns 42. Both the first lens 3 and the second lens are prime lenses, and the first set of focusing lens 33 and the second set of focusing lens 43 are focused by the focusing ring 6.

Both the first lens 3 and the second lens 4 are integrated into the insert tube 1. An illumination optical fiber 21 is filled between the outside faces of the protective casings of the first lens 3 and the second lens 4 and the inside face of the front tube 11. The illumination optical fiber 21 is communicated with an external light source through an optical fiber interface 22, so that the illumination light is projected from a port at the inserted end for illuminating the surgery field. The rear tube 12 is formed with external threads 121 on an outer side of one end thereof away from the inserted end of the insert tube 1, that is, the insert tube 1 is formed with external threads 121 at one end thereof.

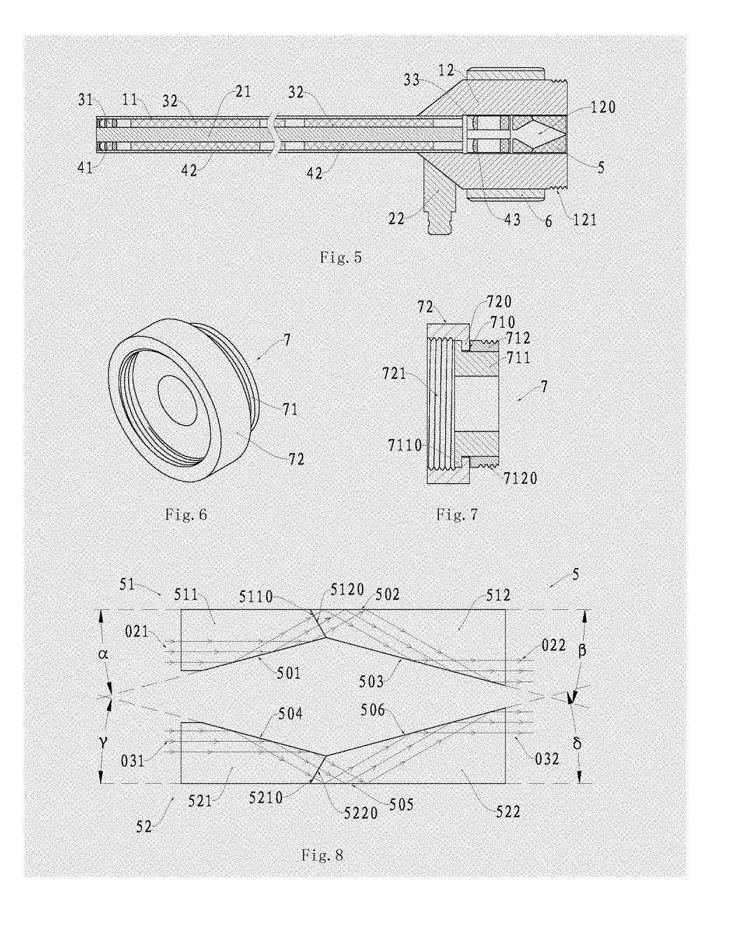

Referring to FIGS. 6 and 7, the connecting assembly 7 comprises a fixed connecting ring 71 and a rotary connecting ring 72. The fixed connecting ring 71 comprises an inner ring 711 and an outer ring 712 sheathed outside the inner ring 711. The rotary connecting ring 72 is protruded to form an annular bulge 720 on an inner side of one end thereof close to the fixed connecting ring 71, and the inner ring 711 is protruded to form an annular bulge 7110 on an outer side of one end thereof close to the rotary connecting ring 72. After the outer ring 712 is sheathed outside the inner ring 711 and fixedly connected to the inner ring 711, an annular groove 710 for accommodating the annular bulge 720 is formed between an end face of the annular bulge 7110 and an end face of the outer ring 712, so that one end of the rotary connecting ring 72 is buckled onto one end of the fixed connecting ring 71 and can rotate about its own axis relative to the fixed connecting ring 71. The outer ring 712 is form with external threads 7120 matched with the internal threads 011 shown in FIG. 1 on an outer side of one end thereof away from the rotary connecting ring, and the rotary connecting ring 72 is form with internal threads 721 matched with the external threads 121 shown in FIG. 5 on an inner side of one end thereof away from the fixed connecting ring 71. The direction of turning of the internal threads 721 is opposite to that of the external threads 7120.

Referring to FIG. 8, the optical deflection assembly 5 comprises a first prism 51 and a second prism 52. The first prism 51 is formed by gluing a first front prism 511 and a first rear prism 512. Anti-reflection films are coated on both a gluing surface 5110 of the first front prism 511 and a gluing surface 5120 of the first rear prism 512. The second prism 52 is formed by gluing a second front prism 521 and a second rear prism 522. Anti-reflection films are coated on both a gluing surface 5210 of the second front prism 521 and a gluing surface 5220 of the second rear prism 522.

Referring to FIGS. 3, 5 and 8, in a travelling direction of light beams in the endoscope, the first prism 51 is located downstream of the first lens 3, and the second prism 52 is located downstream of the second lens 4.

The first lens 3 receives and projects a first light beam 021, and the first light beam 021 is projected onto an incident surface of the first prism 51. After the first light beam 021 enters the first front prism 511, it is reflected by a first reflection plane 501 and projected from the gluing surface 5110, then enters the first rear prism 512 from the gluing surface 5120, and is successively reflected by a second reflection plane 502 and a third reflection plane 503 and eventually projected from an emergent surface of the first rear prism 512 to form a first light beam 022.

The second lens 4 receives and projects a second light beam 031, and the second light beam 031 is projected onto an incident surface of the second prism 52. After the second light beam 031 enters the second front prism 521, it is reflected by a fourth reflection plane 504 and projected from the gluing surface 5210, then enters the second rear prism 522 from the gluing surface 5220, and is successively reflected by a fifth reflection plane 505 and a sixth reflection plane 506 and eventually projected from an emergent surface of the second rear prism 522 to form a second light beam 032.

In the first prism 51, the second reflection plane 502 is parallel to an optical axis of the first light beam 021. In the travelling direction of light beams in the endoscope, the spacing between the first reflection plane 501 and the second reflection plane 502 decreases gradually, the spacing between the third reflection plane 503 and the second reflection plane 502 increases gradually, and an included angle α between the first reflection plane 501 and the second reflection plane 502 is equal to an included angle β between the third reflection plane 503 and the second reflection plane 502. In the second prism 52, the fifth reflection plane 505 is parallel to an optical axis of the second light beam 031. In the travelling direction of light beams in the endoscope, the spacing between the fourth reflection plane 504 and the fifth reflection plane 505 decreases gradually, the spacing between the sixth reflection plane 506 and the fifth reflection plane 505 increases gradually, and an included angle γ between the fourth reflection plane 504 and the fifth reflection plane 505 is equal to an included angle δ between the sixth reflection plane 506 and the fifth reflection plane 505.

All the first reflection plane 501, the second reflection plane 502, the third reflection plane 503, the fourth reflection plane 504, the fifth reflection plane 505 and the sixth reflection plane 506 are formed by coating reflective films on surfaces of the prisms. By the reflection of these reflection planes, the spacing between the optical axes of the first light beam and the second light beam is changed.

Referring to FIG. 9, a method for upgrading an existing two-dimensional endoscope system to a three-dimensional endoscope system by using the endoscope described above includes a selection step S1, a butt-jointing and limiting step S2, a step S3 of upgrading and replacing software and a step S4 of upgrading and replacing a display device. The step S3 of upgrading and replacing software and the step S4 of upgrading and replacing a display device form a step of upgrading and replacing the rear-end processing device in this embodiment.

In the selection step S1, an endoscope matched with the camera connector 01 is selected, including: (1) a mechanical connection interface of a connecting assembly in an optical machine connecting unit is matched with a mechanical connection interface of the camera connector 01; (2) since a first light beam and a second light beam projected by a first lens and a second lens will not be matched with the dimension of the image sensor, it is necessary to select an appropriate optical deflection assembly to change the spacing between optical axes of the first light beam and the second light beam, so as to satisfy the requirements for the dimension of the image sensor; and (3) the spacing between the optical deflection assembly and the image sensor is limited by using the connecting assembly, so as to satisfy the requirements for the spacing between the two, specifically as follows:

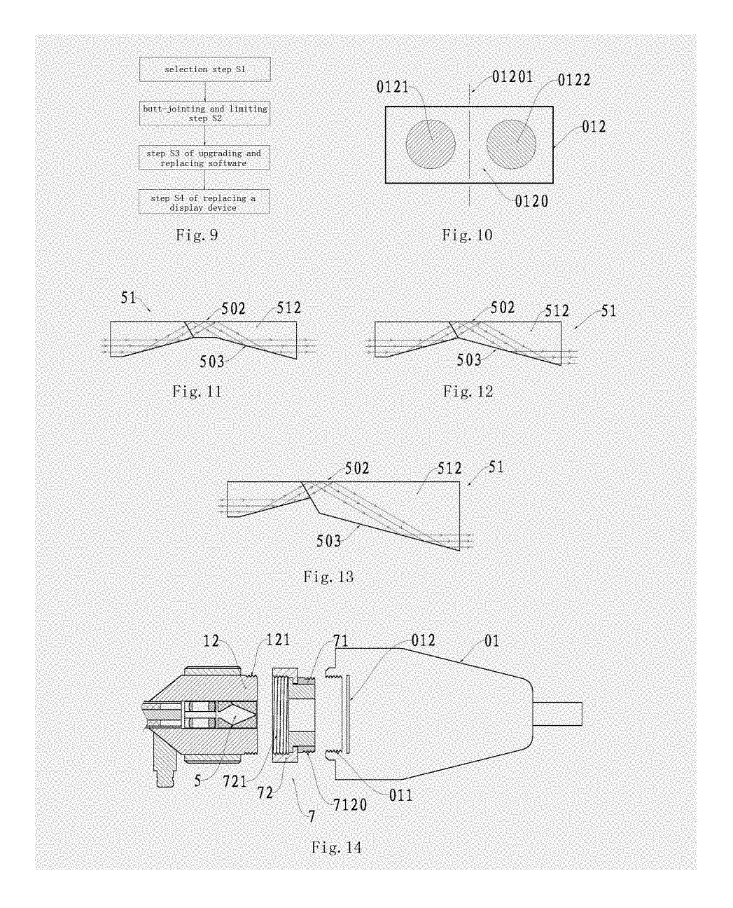

Referring to FIG. 10, in accordance with the requirements for the dimension of the image sensor 012, in a widthwise direction, a target surface 0120 of the image sensor 012 is divided into two portions. A first photosensitive region 0121 is provided in the left half portion, and a second photosensitive region 0122 is provided in the right half portion. The first photosensitive region 0121 and the second photosensitive regions 0122 are two regions that are separated from each other, that is, there is no overlap between the two.

As shown in FIG. 8, by selecting an appropriate optical deflection assembly, the first light beam 022 projected by the first prism 51 is projected onto the first photosensitive region 0121, and the second light beam 032 projected by the second prism is projected onto the second photosensitive region 0122. Therefore, the selection of the first prism 51 and the second prism 52 of different dimensions and structures is needed, as shown FIGS. 11-13, the selection of the dimension and structure of the prism will be described below by taking the first prism 51 as example.

When the longitudinal dimension of the first rear prism 512 satisfies the requirements, if not, adjustments can be made according to actual needs, by changing the spacing between the third reflection plane 503 and the second reflection plane 502, the relative position between optical axes of the projected first light beam and the incident first light beam can be adjusted, so that the spacing between the optical axes of the first light beam and the second light beam is changed. It is also possible that the spacing between the optical axes of the first light beam and the second light beam is changed by changing the included angle between first reflection plane and the second reflection plane and the included angle between the third reflection plane and the second reflection plane, so that the imaging requirements for the image sensors of different dimensions are satisfied.

In the butt-jointing and limiting step S2, referring to FIG. 14, the endoscope is butt-jointed with the camera connector 01 through the connecting assembly 7, and the spacing between the optical reflection assembly 5 and the image sensor 012 in the camera connector 01 is limited. Specifically as follows:

The endoscope is fixed by a first fixture, the camera connector 01 is fixed by a second fixture, and the relative position relationship between the optical deflection assembly 5 and the image sensor 012 is adjusted well. At least one of the first fixture and the second fixture can be axially moved along the insert tube. The external threads 7120 on the fixed connecting ring 71 are fixedly connected to the internal threads on the camera connector 01 through threads, and the rotary connecting ring 71 is rotated about its own axis relative to the fixed connecting ring 71. By matching the internal threads 721 on the rotary connecting ring 72 with the external threads 121 on the rear tube 12, the spacing between the endoscope and the camera connector 01 is decreased gradually until an end face of the rear tube 12 is rested against an end face of the fixed connecting ring 71 away from the camera connector 01. Since the direction of turning of the internal threads 721 is opposite to the direction of turning of the external threads 7120, the fixed connection among the endoscope, the connecting assembly 7 and the camera connector 01 is realized. Accordingly, while the butt-joint of the endoscope with the camera connector 01 is realized, the spacing between the optical deflection assembly 5 and the image sensor 012 is limited.

In the step S3 of upgrading and replacing software, the original two-dimensional image processing software is updated to and replaced with the three-dimensional image processing software.

In the step S4 of upgrading and replacing the display device, the display device is upgraded to and replaced with a three-dimensional display device having a three-dimensional display function.

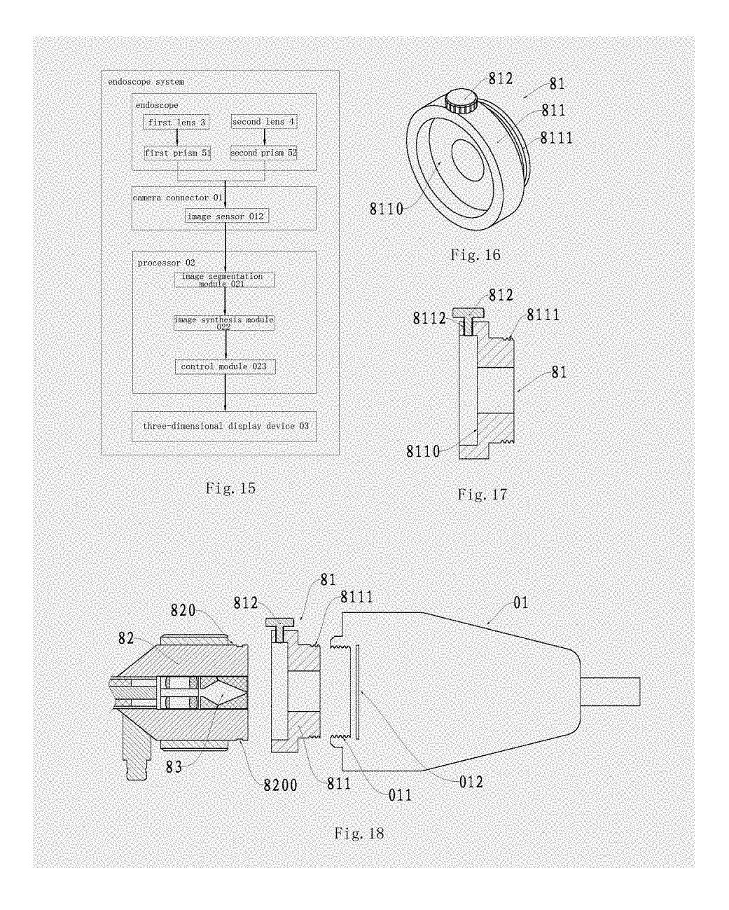

Referring to FIGS. 13-15, a process of operating the modified endoscope system includes an imaging step, a segmentation step, a synthesis step and a developing step.

In the imaging step, a first image of a scene acquired by the first lens 3 and a second image of the same scene acquired by the second lens 4 at the same moment are reflected by the first prism 51 and the second prism 52, so that the spacing between optical axes of a light beam in the first image and a light beam in the second image is changed. By receiving the first image by the first photosensitive region 0121 on the target surface 0120 of the image sensor 012 and synchronously receiving the second image by the second photosensitive region 0122 on the target surface 0120 of the image sensor 012, a third image is generated.

In the segmentation step, the third image obtained in the imaging step is segmented into two two-dimensional images having a parallax therebetween by an image segmentation module 021 in a processor 02.

In the synthesis step, in an image synthesis module 022 of the processor 02, the two two-dimensional images having a parallax therebetween segmented in the segmentation step are processed and synthesized to form a three-dimensional image.

In the developing step, according to the image information recorded in the three-dimensional image synthesized by the image synthesis module 022, a control module 023 in the processor 02 controls the three-dimensional display device 03 to display a three-dimensional image.

### Second Embodiment of the Endoscope

In the description of the second embodiment of the endoscope according to the present invention, only the difference between this embodiment and the first embodiment of the endoscope will be described below.

Referring to FIGS. 16-18, the connecting assembly 81 comprises a connecting collar 811 and a positioning screw 812.

The connecting collar 811 is formed with an inner shoulder 8110 on an inner side of the left end thereof, a screw hole 8112 matched with the positioning screw 812 on a side wall of the left end thereof, and external threads 8111 matched with the internal threads 011 on an outer side of the right end thereof. The right end 820 of the rear tube 82 is fitted with the left port of the connecting collar 81, and an annular positioning groove 8200 matched with the tail of the positioning screw 812 is formed in an outside wall of the right end 820.

In the butt-jointing and limiting step, by fitting the external threads 8111 with the internal threads 011, the connecting collar 811 is fixedly connected to the camera connector 01, the left port of the connecting collar 811 is sheathed outside the right end 820 of the rear tube 82, and an end face of the right end 820 is rested against the inner shoulder 8110. The endoscope is rotated about the axis of the rotary collar 81 relative to the rotary collar 81 until the optical deflection assembly 83 is aligned with the image sensor 012; and, the positioning screw 812 is screwed down, and the tail of the positioning screw 812 is embedded into the annular positioning groove 8200, so that the relative position between the both is fixed.

### Third Embodiment of the Endoscope

In the description of the third embodiment of the endoscope according to the present invention, only the difference between this embodiment and the second embodiment of the endoscope will be described below.

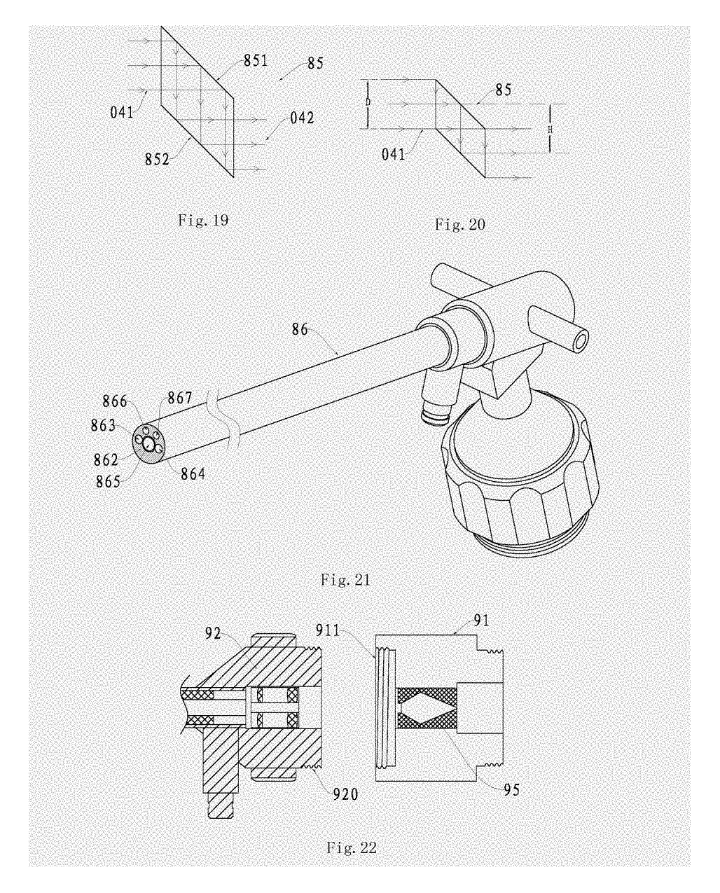

Referring to FIG. 18, the annular positioning groove 8200 is replaced by a positioning hole formed at the right end 820, the positioning screw 812 is a spring plunger, and the rear tube 82 is rotated in place relative to the circumferential direction of the connecting collar 81 by fitting the positioning screw 812 with the positioning hole.

### Fourth Embodiment of the Endoscope

In the description of the fourth embodiment of the endoscope according to the present invention, only the difference between this embodiment and the first embodiment of the endoscope will be described below.

Both the first prism and the second prism are parallelogram prisms 85 shown in FIG. 19. An incident light beam 041 is reflected by a first reflection plane 851 and a second reflection plane 852 and then projected to form an emergent light beam 042. By adjusting the spacing between the optical axis of the incident light beam 041 and the optical axis of the emergent light beam 042, the spacing between the optical axes of the first light beam and the second light beam are adjusted by the first prism and the second prism.

Referring to FIG. 20, the parallelogram prisms 85 are used as deflection prisms. In this embodiment, in a direction perpendicular to an optical axis of a light beam 041, the deflection distance H of the center of the light path is required to be greater than the diameter D of the incident light beam 041.

### Fifth Embodiment of the Endoscope

In the description of the fifth embodiment of the endoscope according to the present invention, only the difference between this embodiment and the first embodiment of the endoscope will be described below.

Referring to FIG. 21, a first lens 863, a second lens 864, an instrument passage 865, a water inlet passage 866, a water outlet passage 867 and an illumination optical fiber 862 are integrated into the insert tube 86. An optical fiber is used instead of the image transmitting column as an image transmitter in this embodiment.

### Sixth Embodiment of the Endoscope

In the description of the sixth embodiment of the endoscope according to the present invention, only the difference between this embodiment and the second embodiment of the endoscope will be described below.

Referring to FIG. 22, the connecting collar 91 is provided with an optical deflection assembly 95 within an inner cavity thereof, the connecting collar 91 is formed with internal threads 911 on an inner side of the left end thereof, and the rear tube 92 is form with external threads 920 matched with the internal threads 911 on an outer side of the right end thereof. When two ends of the connecting collar 91 are fixedly connected to the rear tube 92 and the camera connector through threads, after the threads among the three are tightened in place, the first lens, the second lens, the optical deflection assembly 95 and the image sensor are successively butt-jointed, and the spacing between the optical deflection assembly 95 and the image sensor is limited. The connecting assembly and the optical deflection assembly together form an optical machine connecting device in this embodiment, i.e., an optical machine connecting unit in this embodiment.

### Seventh Embodiment of the Endoscope

In the description of the seventh embodiment of the endoscope according to the present invention, only the difference between this embodiment and the sixth embodiment of the endoscope will be described below.

By fitting the positioning screw provided on the connecting collar and the positioning hole provided on the rear tube, the fixed connection of the connecting collar to the rear tube is realized, and the alignment of the lenses with the prisms is also realized. The first lens and the second lens are not limited to the prime lenses in the above embodiments, and may also be zoom lenses. The fixed connection structure between the connecting assembly and the rear tube are not limited to that descried in the above embodiments, and has various obvious variations. The structure and number of prisms are not limited to that described in the above embodiments, and has various obvious variations.

### Embodiment of the Optical Machine Connecting Device for an Endoscope

The structure of the optical machine connecting device has been described in the sixth embodiment, the seventh embodiment and their combinations with other embodiments of the endoscope, and will not be repeated here.

### Embodiment of the Method for Modifying a Two-Dimensional Endoscope System

The method for modifying a two-dimensional endoscope system has been described in the embodiments of the endoscope and will not be repeated here.

### INDUSTRIAL APPLICABILITY

The endoscope according to the present invention is suitable for a scene of upgrading an existing endoscope system to a three-dimensional endoscope system. By the modifying method of the present invention, the existing two-dimensional endoscope system is upgraded and modified by the product of the present invention. Consequently, the cost for upgrading and modification can be reduced, and the number of idle facilities in the existing two-dimensional endoscope system is decreased.

Free contents in the sequence list

1-10. canceled

11. An endoscope comprising:
an insert tube, comprising:
a first lens integrated into the insert tube; and
a second lens integrated into the insert tube;
an optical machine connecting unit comprising an optical deflection assembly, the optical deflection assembly comprising:
a first prism located downstream of the first lens, in a travelling direction of light beams in the endoscope, wherein the first prism is used for reflecting a first light beam received and projected by the first lens; and
a second prism located downstream of the second lens, in a travelling direction of light beams in the endoscope, wherein the second prism is used for reflecting a second light beam received and projected by the second lens;
wherein a spacing between optical axes of the first light beam and the second light beam is changed, so as to match an image sensor in a camera connector of a two-dimensional endoscope system; and,
a connecting assembly used for forming a butt joint between the endoscope and the camera connector and limiting a spacing between the optical deflection assembly and the image sensor.

12. The endoscope according to claim 11, wherein both the first prism and the second prism are parallelogram prisms.

13. The endoscope according to claim 11, wherein:
the first prism comprises:
a second reflection plane being parallel to the optical axis of the first light beam;
a first reflection plane being located on the same side of the second reflection plane; and
a third reflection plane being located on the same side of the second reflection plane;
wherein, in the travelling direction of light beams in the endoscope, the third reflection plane is located downstream of the first reflection plane, the spacing between the first reflection plane and the second reflection plane decreases gradually, the spacing between the third reflection plane and the second reflection plane increases gradually, and an included angle between the first reflection plane and the second reflection plane is equal to an included angle between the third reflection plane and the second reflection plane;
and,
the second prism comprises:
a fifth reflection plane being parallel to the optical axis of the second light beam;
a fourth reflection plane being located on the same side of the fifth reflection plane; and,
a sixth reflection plane being located on the same side of the fifth reflection plane;
wherein, in the travelling direction of light beams in the endoscope, the sixth reflection plane is located downstream of the fourth reflection plane, the spacing between the fourth reflection plane and the fifth reflection plane decreases gradually, the spacing between the sixth reflection plane and the fifth reflection plane increases gradually, and an included angle between the fourth reflection plane and the fifth reflection plane is equal to an included angle between the sixth reflection plane and the fifth reflection plane.

14. The endoscope according to claim 13, wherein:
the first prism comprises:
a first front prism, on which the first reflection plane is located; and
a first rear prism, on which the second reflection plane and the third reflection plane are located;
wherein butt joint surfaces of the first front prism and the first rear prism are fixedly connected by glue, and anti-reflection films are coated on the butt joint surfaces of the first front prism and the first rear prism;
and,
the second prism comprises:
a second front prism, on which the fourth reflection plane is located; and
a second rear prism, on which the fifth reflection plane and the sixth reflection plane are located;
wherein butt joint surfaces of the second front prism and the second rear prism are fixedly connected by glue, and anti-reflection films are coated on the butt joint surfaces of the second front prism and the second rear prism.

15. The endoscope according to claim 11, wherein:
the connecting assembly comprises:
a positioning screw; and
a connecting collar which is formed with an inner shoulder on an inner side of one end thereof, while the other end thereof is detachably and fixedly connected to the camera connector; wherein the connecting collar is provided with a screw hole matched with the positioning screw on a side wall of the one end thereof;
wherein:
the one end of the connecting collar is sheathed on one end of the insert tube, and the insert tube can rotate about an axis of the connecting collar relative to the connecting collar; and,
when the endoscope is butt-jointed with the camera connector, an end face of the one end of the insert tube is rested against the inner shoulder.

16. The endoscope according to claim 12, wherein the connecting assembly comprises:
a positioning screw; and
a connecting collar which is formed with an inner shoulder on an inner side of one end thereof, while the other end thereof is detachably and fixedly connected to the camera connector;
wherein:
the connecting collar is provided with a screw hole matched with the positioning screw on a side wall of the one end thereof; wherein the one end of the connecting collar is sheathed on one end of the insert tube, and the insert tube can rotate about an axis of the connecting collar relative to the connecting collar; and,
when the endoscope is butt-jointed with the camera connector, an end face of the one end of the insert tube is rested against the inner shoulder.

17. The endoscope according to claim 13, wherein the connecting assembly comprises:
a positioning screw; and
a connecting collar which is formed with an inner shoulder on an inner side of one end thereof, while the other end thereof is detachably and fixedly connected to the camera connector;
wherein:
the connecting collar is provided with a screw hole matched with the positioning screw on a side wall of the one end thereof;
the one end of the connecting collar is sheathed on one end of the insert tube and the insert tube can rotate about an axis of the connecting collar relative to the connecting collar; and,
when the endoscope is butt-jointed with the camera connector, an end face of the one end of the insert tube is rested against the inner shoulder.

18. The endoscope according to claim 14, wherein the connecting assembly comprises:
a positioning screw; and
a connecting collar which is formed with an inner shoulder on an inner side of one end thereof, while the other end thereof is detachably and fixedly connected to the camera connector;
wherein:
the connecting collar is provided with a screw hole matched with the positioning screw on a side wall of the one end thereof;
the one end of the connecting collar is sheathed on one end of the insert tube and the insert tube can rotate about an axis of the connecting collar relative to the connecting collar; and,
when the endoscope is butt-jointed with the camera connector, an end face of the one end of the insert tube is rested against the inner shoulder.

19. The endoscope according to claim 11, wherein the connecting assembly comprises:
a fixed connecting ring, the other end of which is fixedly connected to the camera connector by threads; and
a rotary connecting ring, one end of which is buckled on one end of the fixed connecting ring and can rotate about its own axis relative to the fixed connecting ring;
wherein:
the inner diameter of the fixed connecting ring is less than that of the rotary connecting ring;
the insert tube is formed with external threads on one end thereof, and the rotary connecting ring is formed with internal threads matched with the external threads , on an inside wall of the other end thereof;
the direction of turning of the internal threads is opposite to the direction of turning of the threads formed on the other end of the fixed connecting ring; and,
when the endoscope is butt-jointed with the camera connector, an end face of the one end of the insert tube is rested against an end face of the one end of the fixed connecting ring.

20. The endoscope according to claim 12, wherein the connecting assembly comprises:
a fixed connecting ring, the other end of which is fixedly connected to the camera connector by threads; and,
a rotary connecting ring, one end of which is buckled on one end of the fixed connecting ring and can rotate about its own axis relative to the fixed connecting ring;
wherein:
the inner diameter of the fixed connecting ring is less than that of the rotary connecting ring;
the insert tube is formed with external threads on one end thereof, and the rotary connecting ring is formed with internal threads matched with the external threads on an inside wall of the other end thereof;
the direction of turning of the internal threads is opposite to the direction of turning of the threads formed on the other end of the fixed connecting ring; and,
when the endoscope is butt-jointed with the camera connector, an end face of the one end of the insert tube is rested against an end face of the one end of the fixed connecting ring.

21. The endoscope according to claim 13, wherein the connecting assembly comprises:
a fixed connecting ring, the other end of which is fixedly connected to the camera connector by threads; and
a rotary connecting ring, one end of which is buckled on one end of the fixed connecting ring and can rotate about its own axis relative to the fixed connecting ring;
wherein:
the inner diameter of the fixed connecting ring is less than that of the rotary connecting ring;
the insert tube is formed with external threads on one end thereof, and the rotary connecting ring is formed with internal threads matched with the external threads on an inside wall of the other end thereof;
the direction of turning of the internal threads is opposite to the direction of turning of the threads formed on the other end of the fixed connecting ring; and,
when the endoscope is butt-jointed with the camera connector, an end face of the one end of the insert tube is rested against an end face of the one end of the fixed connecting ring.

22. The endoscope according to claim 14, wherein the connecting assembly comprises:
a fixed connecting ring, the other end of which is fixedly connected to the camera connector by threads; and,
a rotary connecting ring, one end of which is buckled on one end of the fixed connecting ring and can rotate about its own axis relative to the fixed connecting ring;
wherein:
the inner diameter of the fixed connecting ring is less than that of the rotary connecting ring;
the insert tube is formed with external threads on one end thereof, and the rotary connecting ring is formed with internal threads matched with the external threads on an inside wall of the other end thereof;
the direction of turning of the internal threads is opposite to the direction of turning of the threads formed on the other end of the fixed connecting ring; and,
when the endoscope is butt-jointed with the camera connector, an end face of the one end of the insert tube is rested against an end face of the one end of the fixed connecting ring.

23. An optical machine connecting device for an endoscope, the optical machine connecting device being used for connecting the endoscope with a camera connector of a two-dimensional endoscope system, the endoscope comprises an insert tube, the insert tube comprising:
a first lens integrated into the insert tube; and,
a second lens integrated into the insert tube;
wherein:
the optical machine connecting device comprises:
an optical deflection assembly comprising:
a first prism located downstream of the first lens, in a travelling direction of light beams in the endoscope, wherein the first prism is used for reflecting a first light beam received and projected by the first lens; and
a second prism located downstream of the second lens, in a travelling direction of light beams in the endoscope, wherein the second prism is used for reflecting a second light beam received and projected by the second lens; and,
a spacing between optical axes of the first light beam and the second light beam is changed, so as to match an image sensor in a camera connector of a two-dimensional endoscope system;
and,
a connecting assembly used for forming a butt joint between the endoscope and the camera connector and limiting a spacing between the optical deflection assembly and the image sensor.

24. The optical machine connecting device for an endoscope according to claim 23, wherein:
the connecting assembly comprises a connecting collar;
one end of the connecting collar is fixedly connected to one end of the insert tube by threads, while the other end thereof is fixedly connected to the camera connector by threads; and,
both the first prism and the second prism are mounted within the connecting collar.

25. The optical machine connecting device for an endoscope according to claim 23, wherein:
the first prism comprises:
a second reflection plane being parallel to the optical axis of the first light beam;
a first reflection plane being located on the same side of the second reflection plane; and,
a third reflection plane being located on the same side of the second reflection plane;
wherein, in the travelling direction of light beams in the endoscope, the third reflection plane is located downstream of the first reflection plane, the spacing between the first reflection plane and the second reflection plane decreases gradually, the spacing between the third reflection plane and the second reflection plane increases gradually, and an included angle between the first reflection plane and the second reflection plane is equal to an included angle between the third reflection plane and the second reflection plane;
the second prism comprises:
a fifth reflection plane being parallel to the optical axis of the second light beam;
a fourth reflection plane being located on the same side of the fifth reflection plane;
a sixth reflection plane being located on the same side of the fifth reflection plane;
wherein, in the travelling direction of light beams in the endoscope, the sixth reflection plane is located downstream of the fourth reflection plane, the spacing between the fourth reflection plane and the fifth reflection plane decreases gradually, the spacing between the sixth reflection plane and the fifth reflection plane increases gradually, and an included angle between the fourth reflection plane and the fifth reflection plane is equal to an included angle between the sixth reflection plane and the fifth reflection plane.

26. The optical machine connecting device for an endoscope according to claim 24, wherein
the first prism comprises:
a second reflection plane being parallel to the optical axis of the first light beam;
a first reflection plane being located on the same side of the second reflection plane; and,
a third reflection plane being located on the same side of the second reflection plane;
wherein, in the travelling direction of light beams in the endoscope, the third reflection plane is located downstream of the first reflection plane, the spacing between the first reflection plane and the second reflection plane decreases gradually, the spacing between the third reflection plane and the second reflection plane increases gradually, and an included angle between the first reflection plane and the second reflection plane is equal to an included angle between the third reflection plane and the second reflection plane;
the second prism comprises:
a fifth reflection plane being parallel to the optical axis of the second light beam;
a fourth reflection plane being located on the same side of the fifth reflection plane;
a sixth reflection plane being located on the same side of the fifth reflection plane;
wherein, in the travelling direction of light beams in the endoscope, the sixth reflection plane is located downstream of the fourth reflection plane, the spacing between the fourth reflection plane and the fifth reflection plane decreases gradually, the spacing between the sixth reflection plane and the fifth reflection plane increases gradually, and an included angle between the fourth reflection plane and the fifth reflection plane is equal to an included angle between the sixth reflection plane and the fifth reflection plane.

27. A method for modifying a two-dimensional endoscope system, the two-dimensional endoscope system comprising a two-dimensional endoscope, a camera connector and a rear-end processing device, wherein the method comprises:
selecting a three-dimensional endoscope matched with the camera connector to replace the two-dimensional endoscope, the three-dimensional endoscope being the endoscope according to claim 11;
butt-jointing the three-dimensional endoscope with the camera connector by the connecting assembly, and limiting a spacing between the optical deflection assembly and an image sensor in the camera connector; and,
upgrading and replacing the rear-end processing device with a rear-end processing device of a three-dimensional endoscope system.

28. A method for modifying a two-dimensional endoscope system, the two-dimensional endoscope system comprising a two-dimensional endoscope, a camera connector and a rear-end processing device, wherein the method comprises:
selecting a three-dimensional endoscope matched with the camera connector to replace the two-dimensional endoscope, the three-dimensional endoscope being the endoscope according to claim 12;
butt-jointing the three-dimensional endoscope with the camera connector by the connecting assembly, and limiting a spacing between the optical deflection assembly and an image sensor in the camera connector; and,
upgrading and replacing the rear-end processing device with a rear-end processing device of a three-dimensional endoscope system.

29. A method for modifying a two-dimensional endoscope system, the two-dimensional endoscope system comprising a two-dimensional endoscope, a camera connector and a rear-end processing device, the method comprising:
selecting a three-dimensional endoscope matched with the camera connector to replace the two-dimensional endoscope, the three-dimensional endoscope being the endoscope according to claim 13;
butt-jointing the three-dimensional endoscope with the camera connector by the connecting assembly, and limiting a spacing between the optical deflection assembly and an image sensor in the camera connector; and,
upgrading and replacing the rear-end processing device with a rear-end processing device of a three-dimensional endoscope system.

30. A method for modifying a two-dimensional endoscope system, the two-dimensional endoscope system comprising a two-dimensional endoscope, a camera connector and a rear-end processing device, the method comprising the following steps:
selecting a three-dimensional endoscope matched with the camera connector to replace the two-dimensional endoscope, the three-dimensional endoscope being the endoscope according to claim 14;
butt-jointing the three-dimensional endoscope with the camera connector by the connecting assembly, and limiting a spacing between the optical deflection assembly and an image sensor in the camera connector; and,
upgrading and replacing the rear-end processing device with a rear-end processing device of a three-dimensional endoscope system.
